(19) United States
(12) Patent Application Publication
(10) Pub. No.: US 2008/0292597 A1
(43) Pub. Date: Nov. 27, 2008

(54) Umbilical Cord Stem Cell Composition & Method of Treating Neurological Diseases

(76) Inventor: David A Steenblock, San Clemente, CA (US)

Correspondence Address: CONNORS ASSOCIATES, 1600 DOVE ST, SUITE 220, NEWPORT BEACH, CA, 92660, US

(21) Appl. No.: 11/658,327

(22) PCT Filed: Jul. 28, 2005
(86) PCT No.: PCT/US05/26992

§ 371 Date: Jan. 23, 2007

Related U.S. Application Data
(60) Provisional application No. 60/592,167, filed on Jul. 29, 2004.

(51) Int. Cl. A61K 35/12; A61P 25/00

(52) U.S. Cl. 424/93.7; 424/530

(57) ABSTRACT

A neurological disease is treated by administering to a patient a therapeutically effective amount of a composition including human umbilical cord stem cells. The composition may include growth factors mixed with the stem cells immediately prior to being administered. A specific pre and post transplantation protocol provides optimal methods for obtaining favorable clinical results.

### RELATED PATENT APPLICATIONS & INCORPORATION BY REFERENCE

This application is a PCT application which claims the benefit under 35 USC 119(e) of U.S. provisional patent application Ser. No. 60/592,167, entitled “UMBILICAL CORD STEM CELL COMPOSITION & METHOD OF TREATING NEUROLOGICAL DISEASES,” filed Jul. 29, 2004. This related applications is incorporated herein by reference and made a part of this application. If any conflict arises between the disclosure of the invention in this PCT application and that in the related provisional application, the disclosure in this PCT application shall govern. Moreover, the inventor incorporates herein by reference any and all U.S. patents, U.S. patent applications, and other documents, hard copy or electronic, cited or referred to in this application.

### DEFINITIONS

The words “comprising,” “having,” “containing,” and “including,” and other forms thereof, are intended to be equivalent in meaning and be open ended in that an item or items following any one of these words is not meant to be an exhaustive listing of such item or items, or meant to be limited to only the listed item or items.

The words “consisting,” “consists of,” and other forms thereof, are intended to be equivalent in meaning and be closed ended in that an item or items following any one of these words is meant to be an exhaustive listing of such item or items and limited to only the listed item or items.

The words “disease” or “diseases” include any injury, disorder, malady, or other condition of the human body that causes pain or dysfunction of any part of the human body.

### BACKGROUND OF INVENTION

Currently human embryonic stem cells are being studied to treat neurological diseases. Because these stem cells are derived from a human embryo or fetus, there have been objections raised to the use of such materials on ethical and scientific/medical grounds. This invention overcomes this objection by using stem cells derived from an alternate source that is unobjectionable yet effective.

### SUMMARY OF INVENTION

This invention has one or more features as discussed subsequently herein. After reading the following section entitled “DETAILED DESCRIPTION OF ONE EMBODIMENT OF THIS INVENTION,” one will understand how the features of this invention provide its benefits, which include, but are not limited to, providing an effective treatment of neurological diseases, especially cerebral palsy and traumatic brain injury in children, using ethically unobjectionable stem cells derived from human umbilical cord material.

Without limiting the scope of this invention as expressed by the claims that follow, some, but not necessarily all, of its features are:

One feature of this invention is a composition of matter. One embodiment of this composition comprises a mixture of human umbilical cord stem cells and blood plasma. The blood plasma may be derived from a patient undergoing medical treatment employing the composition and mixed with the stem cells immediately prior to treatment. The umbilical cord stem cells and the blood plasma typically are substantially free of red and white blood cells and their antigens. This composition may include amniotic fluid or umbilical cord plasma or both. It may also include a parenteral liquid and one or more amino acid. A single dose of this one embodiment of this composition may include at least substantially 1 million umbilical cord stem cells. Of these 1 million umbilical cord stem cells at least substantially 85 percent of the cells may be selected from the group consisting of CD133+ umbilical cord stem cells, CD44− umbilical cord stem cells, CD45− umbilical cord stem cells, CD34−/45+ umbilical cord stem cells, CD34−/45− umbilical cord stem cells, and mesenchymal umbilical cord stem cells.

Another embodiment is the composition of matter comprises a mixture of human umbilical cord stem cells and growth factors. The human growth factors may be growth factors contained in amniotic fluid or umbilical cord plasma or both, or added to the mixture. They may include HGH (human growth hormone), G-CSF (granulocyte colony stimulating factor, GM-CSF (granulocyte macrophage colony stimulating factor, parathyroid (synthetic or natural) hormone, erythropoietin, stem cell factor, LIF (leukemia inhibitory factor). The human growth factors may be derived from blood plasma or added in, with the umbilical cord stem cells and blood plasma being substantially free of red and white blood cells and their antigens. The growth factors may be mixed with the stem cells substantially immediately prior to administration. The stem cells typically are present in amounts and types that simulate growth factors present in umbilical cord plasma. For example, for this composition at least substantially 1 million umbilical cord stem cells may be in a single dose. This composition may comprise at least substantially 10 volume percent blood plasma and a parenteral liquid, and the parenteral liquid may comprises physiological saline and water, Ringers Lactate, and substantially 5 weight percent dextrose in water. The stem cells may be derived from umbilical cord blood, or Warton's Jelly, or mesenchymal (fibromuscular) stem cells derived from an umbilical cord wall, or a combination of two or more or all three. Like the first embodiment, the stem cells may be selected from the group consisting of CD133+ umbilical cord stem cells, CD44− umbilical cord stem cells, CD45− umbilical cord stem cells, CD34−/45+ umbilical cord stem cells, CD34−/45− umbilical cord stem cells, and mesenchymal umbilical cord stem cells.

Another feature of this invention is a method of treating a neurological disease. This method comprises the step of administering to a patient a therapeutically effective amount of a composition including stem cells derived from an umbilical cord. The method is effective in treating cerebral palsy or traumatic brain injury, especially in young children. The method of this invention may use the compositions discussed above. The composition may be administered intravenously, intra-arterially, intramuscular, subcutaneously, intraperitoneally, intracutaneous, intralymphatically, or directly into the part of the patient's body being treated.

Prior to administering the composition certain measures may be taken. For example, the patient may be given chelation therapy, and growth factors may be administered to the patient. A good practice is at least substantially five days prior to administering the composition, and for at least substantially six months after administering the composition, substantially eliminating in the patient cortisone, steroids, glutamate (MSG), and alcohol. Also, following treatment the patient preferably should be on an organic diet rich in anti-oxidants, and free of pesticides, heavy metals, chemicals, food additives, and food coloring agents. The patient may also be given physical therapy after administration of the composition. Another good practice is prior to administering the composition, substantially eliminating in the patient (a) extraneous infections and inflammations in the patient, (b) to the maximum extent possible, heavy metals, and (c) leaky gut syndrome and gut dysbiosis. The composition typically is administered substantially in the absence of any immuno-suppressant compound or chemotherapeutic agents and may be purified, so it is substantially free of red and white blood cells and their antigens. The composition may be administered intravenously by mixing with parenteral liquid flowing into a vein of the patient, with the umbilical cord stem cells being mixed with blood plasma derived from the patient substantially immediately prior to being administered intravenously.

In this method a single dose may be administered including at least substantially 1 million umbilical cord stem cells of which at least substantially 85% of the cells are selected from the group consisting of CD133+ umbilical cord stem cells, CD44− umbilical cord stem cells, CD45− umbilical cord stem cells, CD34−/45+ umbilical cord stem cells, CD34−/45− umbilical cord stem cells, and mesenchymal umbilical cord stem cells. In one embodiment of this method, cerebral palsy or traumatic brain injury in children under the age of about 13 are treated using a single dose of the composition including at least substantially 1 million stem cells that have been purified and expanded from American Association of Blood Banks (AABB) certified human umbilical cord blood.

These features are not listed in any rank order nor is this list intended to be exhaustive.

### DETAILED DESCRIPTION OF ONE EMBODIMENT OF THIS INVENTION

### General

This invention comprises a method of treating neurological diseases and compositions especially suited for this purpose. In accordance with this invention, a neurological disease, in particular cerebral palsy and other brain injured patients with white or grey matter disease are treated by administering to a patient a therapeutically effective amount of a composition including stem cells derived from human umbilical cord material. The composition may be administered in several different ways, including intravenously, intra-arterially, intramuscular, subcutaneously, intraperitoneally, intracutaneous, intralymphatically, or directly into the part of the patient's body being treated.

The human umbilical cord material may be umbilical cord stem cells derived from umbilical cord blood, or may be mesenchymal (fibromuscular) stem cells derived from an umbilical cord wall, or be the combination of both in certain proportions. As part of this invention, it has been discovered that the umbilical cord blood does not necessarily have to be of the same blood type as the patient. The human umbilical cord material is purified so that it is essentially devoid of other blood cells. The stem cells are isolated from umbilical cord blood donated with the consent of the mother and this blood is safety tested for a panel of infections in accordance with the American Association of Blood Banks (AABB) and FDA recommended standards. The stem cells are isolated from the other blood components, for example, using a conventional magnetic bead separation process that removes red and white cells, platelets, and potential antigens and immune cells. About 300,000 stem cells are generally isolated from one placenta-umbilical cord unit.

A single dose of the composition may include at least 1 million stem cells, typically from about 1.5 to about 9 million stem cells. Of these stem cells, desirably over about 85% or more of these cells is primitive CD133+ cells (CD133+ protein marker). The CD34+ (CD34+ protein marker) may also be present. These primitive CD133+ stem cells have the potential of becoming neurons, glia, endothelial cells, hepatocytes, and osteoblast cells, and they have the potential to initiate formation of new blood vessels that increase delivery of oxygen and nutrients to injured and hypoxic tissue. The stem cells may be utilized fresh, or if frozen (−80 C), may be thawed immediately prior to use. Also of utility as part of this application are human umbilical cord stem cells bearing the following biomarkers: CD44−, CD45−, CD34−/CD45+.

Prior to treatment the patient may undergo an enhancing program. Such a program may include a special organic diet rich in anti-oxidants and free of pesticides, heavy metals, chemicals, food additives, and food coloring agents. This diet is also recommended following treatment. This diet is discussed subsequently in greater detail. The enhancing program may include (1) substantially eliminating in the patient extraneous infections and inflammations, (2) to the maximum extend possible, eliminating in the patient heavy metals, (3) substantially eliminating in the patient leaky gut syndrome and gut dysbiosis, and (4) administering to the patient, prior to, or after treatment, or both, supplemental dosages of growth factors, phlebotomy, leukopheresis, plasmapheresis, anti-oxidants, vitamins, minerals, glutathione, stem cell factor, stem cell mobilizing agents, proliferation agents, graft enhancing agents, MMP (matrix metalloproteinase-9) inhibitors and/or stimulators and cell un- and/or trans-differentiation and differentiation agents. At least five days prior to administering the composition and for at least six months after administering the composition, it is desirable to substantially eliminate in the patient excess steroids, glutamate (MSG), vibration, stress and alcohol.

Growth factors may be given to the patient for two days before stem cell transplantation, on the day of stem cell transplantation and daily for three to six months post-transplantation. Suitable growth factors are identified by the National Institute of Health at its web site “Pubmed.” Such growth factors may include, for example, HGH (human growth hormone), testosterone, estrogen, pregnenolone, DHEA (dehydroepiandrosterone), G-CSF (granulocyte colony stimulating factor, GM-CSF (granulocyte macrophage colony stimulating factor, parathyroid (synthetic or natural) hormone, erythropoietin, stem cell factor, and LIF (leukemia inhibitory factor). Further administration of colony stimulating growth factors may be continued for one to three more days post transplantation to enhance mobilization of the patient's own bone marrow stem cells and to enhance the proliferation of both the newly transplanted exogenous stem cells and the patient's own bone marrow derived endogenous stem cells. Mobilizing agents may also be administered to the patient before and after treatment. For example, the mobilizing agents may be sulfated fucoidans. Proliferating agents, such as, for example, parathyroid hormone (natural or synthetic), may be given for one month prior to transplantation and for two to four weeks after transplantation.

Prior to treatment the patient may take the blood tests listed below to ascertain his or her medical condition. Before proceeding with stem cell transplantation, the results of these blood tests desirably should indicate that the patient is substantially free of heavy metals, hydrocarbons, inflammations, infections, and hormonal imbalances. Treatment may be delayed until the patient's blood tests indicate that the patient is substantially detoxified and substantially free of infections and that all physiological parameters are substantially optimal.

### Blood Tests

a) CBC with differential and platelets

b) Chem Screen Panel, electrolytes, magnesium

c) Lipid Panel (cholesterol, HDL, triglycerides, LDL)

d) ESR

e) CRP, Quantitative

f) DHEA Sulfate

g) Fibrinogen and D-Dimer

h) UA with micro/culture and sensitivity, if indicated

i) T3, T4, TSH

j) ANA

k) Ferritin, Serum Iron, TIBC, % saturation

l) PT, PTT

m) Serum Copper and zinc

n) DMSA Challenge Test

o) Homocysteine

p) Folic Acid (serum and RBC)

q) Urine quantitative organic acid test

r) CDSA—comprehensive digestive stool analysis

s) MMP-9 (matrix metalloproteinase-9)

t) Alpha-2-Macroglobulin

u) Alpha-2-antitrypsin

v) Alpha-1-antitrypsin

w) Euglobulin lysis time

x) GGTP (gamma glutamyltranspeptidase)

Even if desirable blood tests are unobtainable in some patients, the treatment in accordance with this invention may nevertheless provide improvement in such patients' clinical outcome.

Physical therapy may also be beneficial, for example, various physical manipulations may be employed to enhance engraftment such as:

(1) Phlebotomy. Phlebotomy decreases the iron content of the blood, which decreases the oxidative nature of the blood, thus limiting differentiation within the first few weeks after transplantation when the therapeutic effect sought is one of in vivo proliferation. Phlebotomy decreases the red cell content of the blood and lowers the available oxygen to the affected tissues, which stimulates the expression of endothelial stem cell adhesion molecules in the affected tissues. Phlebotomy also stimulates the natural release of erythropoietin from the patient's kidneys, which stimulates and promotes the proliferation of the stem cells in vivo after they have been administered;

(2) Electromagnetic stimulation of the affected area prior to transplantation to enhance endothelial cell adhesion molecule expression;

(3) Ultrasound therapy applied to the affected tissue areas to enhance endothelial cell adhesion molecule expression prior to transplantation;

(4) Generalized or local hyperthermia treatments to the affected areas or whole body to enhance stem cell endothelial cell adhesion molecule_expression;

(5) Infrared, ultraviolet, x-ray, gamma ray generalized or local treatment to the affected areas or whole body to enhance stem cell endothelial cell adhesion molecule expression;

(6) Surgical or physical manipulation, dissection or abrasion of the affected tissues to enhance stem cell endothelial cell adhesion molecule expression;

(7) Magnetic beads either by themselves or combined with stem cells or antibodies directed toward the affected tissues and/or their components to enhance stem cell endothelial cell adhesion molecule expression; and

(8) A combination of the above-mentioned physical manipulations or another physical therapy in combination with one or more of the above-mentioned physical manipulations.

### Composition

One embodiment of the composition of this invention is a mixture of umbilical cord stem cells and non-human or human derived growth factors mixed with the stem cells immediately prior to transplantation of the stem cells in the patient. Desirably the amounts and types of growth factors simulate those present in human umbilical cord plasma. In one embodiment, the composition includes blood plasma derived from the patient being treated. Additionally, the composition of this invention may include amniotic fluid or umbilical cord serum or both. One dose of the composition of this invention comprises at least about 1 million umbilical cord stem cells. It may include at least about 10 volume percent blood serum and a parenteral liquid. For example, one embodiment of the composition may comprise from about 1.5 to about 9 million umbilical cord stem cells, from about 10 volume percent blood serum, and from about 90 volume percent parenteral liquid. A suitable parenteral liquid includes, for example, physiological saline and water, Ringers Lactate, and 5 weight percent dextrose in water. The composition is usually administered in the absence of any immuno-suppressant compound or toxic chemotherapeutic agent(s).

### Pre-Treatment Protocol for Stem Cell Therapies

While each treatment protocol will depend on the disease and individual involved, some general guidelines for stem cell pre-treatment are as follows:
1. Stem cells work best when and where acute tissue damage has or is occurring. Ischemic and disrupted blood vessels produce strong cell signaling that induces stem cell migration and proliferation into the target tissue/organs.
2. If possible, all other extraneous infections/inflammatory sites should be treated before stem cell therapy to avoid the stem cells being diverted away from the primary target that is being treated or being differentiated by various cytokines and other oxidizing agents.
3. Heavy metals (lead, cadmium, mercury, arsenic, etc) are injurious to stem cells and should be reduced through oral or IV chelation to the point where no excess heavy metals can be identified after a DMSA (Dimercaptosuccinic acid), or other appropriate chelating agent challenge test.
4. Leaky gut syndrome and gut dysbiosis should be treated to prevent toxins (mesh definition) from entering the body and interfering with the action of the stem cells.
5. Cortisone, steroids, glutamate (MSG), vibration, stress, increased oxygen levels such as provided by supplemental Hyperbaric Oxygen, other oxidative agents and alcohol exhibit toxic actions on stem cells or their newly produced vasculature and should be minimized for at least five (5) days prior to the administration of stem cells and for six months after the treatment.
   As discussed above, the blood tests (a) to (x) above desirably should be conducted while the others listed below are optional as clinically indicated.

y) Pregnenolone

z) IGF-1

aa) PSA for all men over 40

bb) RBC copper

cc) Ceruloplasmin

dd) Haptoglobin

ee) Glycohemoglobin

ff) Antiphospholipid syndrome panel

yy) Viral Tests—Herpes I, II, VI; Epstein-Barr panel, CMV IgG, IgM, etc. as needed for the condition being treated.

Further laboratory testing may be identified for the individual patient as determined by their physician.

Months to weeks prior to the administration of umbilical cord stem cells, the patient performs a urine heavy metal test using DMSA or other appropriate chelating agent. This test is desirable because heavy metals induce differentiation of stem cells and this stops their proliferation, engraftment and conversion into tissue that is to be repaired or affected. The patient is provided with a kit containing: Blue-top urine tube, styrofoam box, cardboard mailer, Ziplock bag, requisition form, plastic collection jug, DMSA capsule(s). The kit includes these patient instructions.

Patient Instructions
1. Perform this test on first arising in the morning. Immediately after awaking, empty the bladder completely. Do not eat any food for one hour. Drink only a moderate amount of fluids.
2. Take the provided 500 mg DMSA capsule with an 8-ounce glass of water immediately after emptying the bladder (For children—3.3 mg DMSA per pound of body weight).
3. Collect all urine in the plastic collection jug for six hours after talking the DMSA capsule. The patient may eat breakfast one hour or more after taking the DMSA capsule. Drink only a moderate amount of fluid during the 6-hour collection period. Excessive fluid will dilute the urine and might reduce the accuracy of the test.
4. At the end of the 6 hours, empty the bladder of all urine into the collection jug one last time. Mix the jug well (swish around or shake gently) and fill the smaller blue-top tube with urine from the jug to mail to the laboratory.
5. Place the blue-top tube in the Ziploc bag provided and then into the Styrofoam container. Make sure the cover is screwed on tightly so that it does not leak.
6. Place the Styrofoam container in the cardboard mailer and send to the medical laboratory at the address on the box.
7. If not mailing the sample on the same day, please keep the sample refrigerated until mailed.
8. Please fill out the requisition form completely, indicating the number of silver-amalgam metal dental fillings. Gold and porcelain fillings do not count. If not sure how many, make an estimate.
If any questions, please call the physician.
   Results from this test may indicate acute or chronic excessive heavy metal accumulation within the patient's body has occurred.

### EXAMPLE

### Stem Cell Administration Techniques

### Pretreatment

For 1 to 3 weeks (5 days a week) prior to treatment the patient undergoes a daily EDTA (ethylene-diamine-tetra acetic acid) chelation therapy. This lowers the blood level of calcium, removes heavy metals and elevates the parathyroid hormone level. Calcium in excess is well recognized as being responsible for “the common final pathway of cell death.” If a cell is injured for any reason, the cell membrane becomes leaky. There are 10,000 molecules of calcium outside of each cell of the body to one molecule present within each cell. When the cell membrane is injured, calcium floods into the cell through the damaged cell wall. As calcium enters the cell in excess, the first thing that happens is the formation of a shell of calcium around the mitochondria (power plants of the cell) that prevents glucose from entering the mitochondria. This interrupts the production of energy within the cell and the cell gradually loses its energy, falls apart and dies. Neurological diseases such as brain injury, ischemia (poor blood flow), Alzheimer's, Parkinson's, Multiple Sclerosis, etc. are associated with an excess of extra-cellular calcium. These and many other chronic degenerative disease conditions are usual improved by EDTA chelation therapy since it removes this excess extra-cellular calcium.

By removing excess calcium from the body's tissues, a hormonal reaction occurs in that a certain hormone called parathyroid hormone is produced. This hormone liberates calcium from any place in the body that calcium is stored including all of the soft tissues where calcium has previously precipitated as a result of either acute or chronic injuries. This is the calcium found in atherosclerotic arteries and arthritic joints and the calcium that accumulation in and around damaged brain cells and furthers their progressive deterioration.

In conjunction with the EDTA clinical, experience has shown that 30 to 120 ml of 3% to 8.5 weight % mixed amino acids included as part of the parenteral fluid being administered provides the stem cells with adequate amino acids in the patient's serum which allows optimum proliferative activity and maximizes the clinical results.

For those unable to do intravenous EDTA chelation therapy, a prescription for a month supply of Forteo® (human teriparatide parathyroid hormone) is recommended. This drug has recently been approved by the FDA as a treatment for osteoporosis. The usual dosage of Forteo® is one subcutaneous injection per day of 20 micrograms. Younger patients and patients under 120 pounds may take this dosage for one month. Older individuals weighing more that 120 pounds should take two injections per day for two weeks. This hormone is one of the best stimulators of stem cell multiplication and growth known. The use of parathyroid hormone may be started on the same day that the administration of growth factors, for example, G-CSF, is started. The combination of daily EDTA treatments prior to stem cell administration and the use of parathyroid hormone injections thereafter may be used to optimize further the clinical benefits.

Beginning one week or more before administering the stem cells, the patient may begin taking one or two units of HGH subcutaneously in the evening daily. This may be continued for the next six months to facilitate the growth of new tissues. Beginning four to five days prior to stem cell administration, G-CSF (neupogen) 10 microgram per kilogram body weight is administered daily subcutaneously, and on the third to fifth day (the day of treatment) is administered in the morning. The stem cells are then administered between four and six hours later. During the same time a therapeutic dosage of mixed fucoidan sulfates are given orally to facilitate the mobilization of endogenous bone marrow stem cells as well as to keep the exogenous stem cells from premature engraftment while in their proliferative phase.

At the time of stem cell administration, the physician has the option of removing 40 to 400 milliliters of blood. This blood is used for processing in the administration of stem cells and the rest may be discarded. The decision as to where phlebotomy is done or not is dependent on the ferritin level in combination with red cell count, the hemoglobin, the serum iron, total iron binding capacity and the percent saturation, the patient's overall condition and the condition of the patient's veins.

### Treatment

The following protocol describes making an aqueous solution of 10-20 volume % serum, 0-10 volume % glutathione, and 80 volume % Ringers Lactate. This solution is mixed with stem cells quickly after the thawing of frozen stem cells. Although in this protocol glutathione is mixed with the Ringers Lactate, this is optional. This solution containing the stem cells is given intravenously to the patient.

### Protocol for Stem Cell IV Administration

Starting with a 100 TO 500 milliliter (ml) container of Ringers Lactate, 5% by weight dextrose in water or 0.9% by weight saline (the container preferably is a glass bottle but plastic container may be used), drain off and discard 10-20%. This leaves 80-90% of the Ringers Lactate remaining in the container. Hook up a universal intravenous (IV) administration set with one Y access port and extra access ports and run out a small amount, for example, 10 ml of the Ringers Lactate from the IV set's plastic discharge tube and then discontinue the flow from the discharge tube using a roller shut off valve provided with the set. Add 1 to 10 ml of an aqueous solution of 100 milligrams (mg) of glutathione per ml of water into the 80-90 ml of the remaining Ringers Lactate for a total of 100 ml (herein Ringers Lactate/Glutathione solution). Other growth factors such as G-CSF may be added in small quantities at this time to facilitate stem cell viability upon thawing. Add one to four vials of mannitol to the Ringer's lactate to enhance opening of the blood brain barrier. The universal intravenous (IV) administration set, a 20 or 21 gauge butterfly ¾ inch Vacuflow, and a safe multi-sample blood collection set with Luer adapter inserted into the largest possible accessible vein is used to administer to the patient the stem cells in accordance with this invention. Heparin may be added at this point to the IV container in amounts determined by the physician deemed necessary to insure no blood clotting processes occur during this procedure which would interfere with the distribution of the stem cells.

Blood is withdrawn from the patient by inserting a vacutainer into the Vacuflow collection set needle. Blood is drawn into four (4) separate 10 ml red, plain vacutainer glass tubes. Remove from the end of the vacutainer's butterfly connection, the vacutainer's needle which has just been used for transferring blood into the glass vacutainer tubes and insert the end of the discharge tube of the previously filled IV set into the now open end of the vacutainer butterfly connection. Now begin the intravenous injection of the Ringers Lactate/Glutathione solution, running this solution slowly to just keep the patient's vein open.

While the Ringers Lactate/Glutathione/mannitol solution is infusing, the patient's blood serum is separated from the collected blood specimens in the four glass tubes. First, spin each of the 4 blood-filled vacutainer tubes down in a centrifuge at 5000 revolutions per minute (rpms) for a full 15 minutes. After centrifuging, remove the serum from each of the 4 serum/blood-filled tubes. With a 3 ml syringe attached to a 1.5 inch 20 gauge needle withdraw 1 ml of serum from one of the tubes, replacing the needle guard after withdrawal. With a 20 ml clean sterile syringe attached to a 1.5 inch 20 gauge needle, remove as much as possible of the remaining serum from each of the tubes. If at least 10 milliliters cannot at this time be collected, spin again the serum/blood-filled tubes for another 10-15 minutes to further separate the serum from the serum/blood mixture. Don't use any serum that has become blood tinged and won't clear on centrifugation. At this time there is 10 ml or more of yellow, clear serum in the 20 ml syringe and 1 ml of serum in the 3 ml syringe.

A mixture of the Ringers Lactate/Glutathione solution and the patient's blood serum is now prepared by adding the 10 ml of serum from the 20 ml syringe to the IV container holding the Ringers Lactate/Glutathione solution to make a mixture that is at least a 10 volume % serum. (herein Serum/Ringers Lactate/Glutathione solution). If less than 10 ml of the patient's serum is collected, for example, only 5 ml, just drain the IV container down into the patient until 30 to 50 ml of Ringers Lactate/Glutathione solution remains in the IV container and then add the appropriate volume of serum to this container to prepare a Serum/Ringers Lactate/Glutathione solution containing at least 10 volume % serum. In other words, it is desirable to use a Serum/Ringers Lactate/Glutathione solution containing at least 10 volume % of the patient's serum. Before adding stem cells to the Serum/Ringers Lactate/Glutathione solution, run into the patient 10 ml of this solution through the IV plastic discharge tube so that the serum adsorbs onto the internal surface of the plastic tube. This helps prevent stem cells from adhering to the plastic tube and not passing into the patient.

Withdraw from the IV container 10 ml of the Serum/Ringers Lactate/Glutathione solution into another 20 ml syringe with a 1.5 inch 20 gauge needle and put this second 20 ml syringe aside. The Serum/Ringers Lactate/Glutathione solution is allowed to run into the patient until there is 30 to 50 ml of this solution remaining in the IV container. If initially only 50 ml of the Serum/Ringers Lactate/Glutathione solution is available, then run into the patient 10 milliliters to cover the inside of the tubing after which time, run the solution in slowly to keep the patient's vein open until there is from 20 to 30 ml of this solution remaining in the IV container. Check the IV needle insertion carefully by lowering the bag below the patient's body to see if a good back flow occurs, indicating that there is a good IV connection (This will help insure that the stem cells will not be deposited into subcutaneous tissue).

At this point in the procedure the following has been prepared:

(1) One 20 ml syringe with a 20 gauge 1.5 inch needle containing 10 ml of the Serum/Ringers Lactate/Glutathione solution (10%+serum).

(2) One 3 ml syringe with 1 milliliter of the patient's serum.

(3) The IV container connected to the patient containing from about 30 to about 50 ml of the Serum/Ringers Lactate/Glutathione solution (10%+serum).

The stem cells are now prepared. Acceptable stem cells may be maintained in a frozen state in 3 ml cryovials vials retained in a dry ice storage receptacle at about −79 degrees Centigrade. About 1.5 grams (about 1.5 million cells) of stem cells are in each cryovial. Remove one vial from the dry ice storage receptacle using a glove or dry ice/liquid nitrogen pickup instrument. Place the vial of stem cells directly into a 37 degree Centigrade incubator. After about 30 seconds, look through the vial into its interior. Watch intermittently as the frozen cells melt. Do not agitate or shake the vial at any time. As the cells melt and reach a condition whereby a very tiny piece of the cell mass is still present, withdraw the vial from the 37 degree Centigrade incubator, unscrew its top, insert the opened vial into a wooden block holder, and then slowly drop-by-drop add 1 ml of serum to the stem cell vial from the 3 ml syringe. When the serum-cell mix is about ¼ inch from the vial's open top, stop adding the serum and place the syringe down on a counter with its needle guard back in place over the needle.

Take the 20 ml syringe filled with 10 ml of Serum/Ringers Lactate/Glutathione solution and insert the end of its attached 20 gauge 1.5 inch sterile needle into the bottom of the vial containing the thawed stem cells, and carefully and slowly withdraw the stem cells into the 20 ml syringe to prepare a mixture of the stem cells and the Serum/Ringers Lactate/Glutathione solution (herein Stem Cells/Serum/Ringers Lactate/Glutathione solution). Once the stem cell vial is empty, wash it by refilling the empty vial with 2 ml of the Stem Cells/Serum/Ringers Lactate/Glutathione solution and again carefully remove everything from the vial with the same 20 ml syringe.

Next, using the 20 ml syringe containing the Stem Cells/Serum/Ringers Lactate/Glutathione solution, insert the needle of the syringe slowly into an Y access port (i.e., the silicone rubber enlarged stopper) of the IV set and inject slowly the Stem Cells/Serum/Ringers Lactate/Glutathione solution. This should take about 10 minutes. Do not allow the stem cells to move up the IV discharge tube to the IV set's drip chamber.

When finished injecting into the patient the Stem Cells/Serum/Ringers Lactate/Glutathione solution allow 10 ml of the Serum/Ringers Lactate/Glutathione solution to flush the discharge tube of stem cells into the patient, open the roller shut off valve and hold the syringe so only a little liquid (about 10 ml) of the Serum/Ringers Lactate/Glutathione solution in the IV container flows backwards into the syringe. In other words, after 5 minutes of allowing the stem cell fluid to wash into the patient, the syringe is refilled. After the syringe re-accumulates about 15 ml of Serum/Ringers Lactate/Glutathione solution from the IV container, slowly inject this fluid back into the patient the same way as injecting the Stem Cells/Serum/Ringers Lactate/Glutathione solution. If there were any residual cells in the syringe or the IV tube, this washes them out. This syringe may now be removed and discarded. Allow the Serum/Ringers Lactate/Glutathione solution to run in completely and then disconnect.

It is desirable for the patient to adjust his or her diet and lifestyle to enhance the medical treatment of this invention. The following are guidelines suitable for most patients to follow.

### Patient Dietary & Lifestyle for the Period Immediately Following Stem Cell Therapy

With serious cases such as stroke and traumatic brain injury, several stem cell treatments may be required, from once a month to once every six months, depending on the severity of damage.

### The First Four Weeks of the Program

For the first 30 days, the focus is on assisting the stem cells to multiply (proliferate) in vivo as much as possible. The following are factors reported to inhibit or promote stem cell proliferation.
Factors that can Inhibit Stem Cell Growth and Need to be Avoided
Following stem cell therapy, the patient is advised to maintain a strict adherence to the stem cell promoting dietary and lifestyle guidelines (below) for the first week and to avoid the following after treatment (unless otherwise noted):
1) Avoid emotional or physical stress, including strenuous exercise, excitability, depression or extreme temperatures (hot or cold) in showers, baths or weather conditions. Emotional and physical stress increases adrenal hormone levels and can possibly aggravate existing impaired blood flow (if any exists), both of which can interfere with stem cell proliferation.
2) Avoid undue exposure to high intensity household and environmental electromagnetic fields, including direct prolonged sunlight, cell phones, extended time around high power electrical lines, television and computers. These could potentially increase oxidative stress (cell damaging free radical production) and thus inhibit stem cell proliferation. This applies only to the first 3-7 days following stem cell therapy.
3) No papaya and pineapple. These foods contain protein-digesting enzymes that break down proteins in cells and anti-proliferative factors.
4) No onions, garlic, ginger, apples, berries (cranberries, raspberries, blueberries, blackberries, etc), citrus fruits, honey, beer (hops), red wine, cauliflower, broccoli, Brussels Sprouts, and almonds. These foods contain compounds that may interfere with stem cell proliferation. This restriction applies for 1 week following stem cell therapy. These foods can be resumed in moderation after 1 week.
5) No sugars, sweets, candies, carrot juice, or fruit juices. These substances can promote blood sugar highs and lows that can bring about the release of adrenalin (stress response) and oxidative damage, especially to stem cells.
6) No monosodium glutamate. MSG and “hydrolyzed protein” are toxic to new neurons. Additives that contain MSG may be labeled as including hydrolyzed vegetable protein, hydrolyzed protein, hydrolyzed plant protein, plant protein extract, sodium caseinate, calcium caseinate, yeast extract, textured proteins, autolyzed yeast, or hydrolyzed oat flour. MSG may also be included in malt extract, malt flavoring, bouillon, broth stock, artificial or natural flavoring, natural beef or chicken flavoring, seasoning, or spices.
7) Avoid smoking/passive smoke, infections, inflammations, trauma, pollution, etc. These factors magnify any existing blood flow impairments (which results in a lack of blood supply and oxygen) and oxidative stress (Generation of cell-damaging free radicals).
8) No alcohol. It inhibits nerve growth factor and is toxic to new nerve cell growth.
9) No Steroids (including glucocosteroids), medications containing opiates or foods that generate exorphins. These substances are reported to interfere with stem cell growth. Patients with ALS, MS and other neurological disorders are especially advised to refrain from exorphin-generating foods* such as grain, cereals, and milk that can promote allergies, inflammation and subsequent nerve damage. Some people will need to continue their cortisone-like drugs, so check with the physician. (*Exorphins are opioid-like compounds generated during the processing of grains, cereals, etc.)
10) No herbs or herbal medicines, unless prescribed by a physician. Herbs contain a wealth of compounds the nature of which has not yet been fully explored by scientists. Some of these plant chemicals are cytotoxic (meaning they indiscriminately kill cells). As such, it is strongly recommended that all such supplements be stopped for 1 month (The timeframe during which stem cell migration, engraftment and proliferation takes place.)
11) The effect of many vitamins and antioxidants on stem cell activity is unknown. As such, it is best to “err on the side of caution” and stop using all such supplements (save for those specifically recommended herein or by your physician)
   A good general rule is to treat the patient as if he or she is in the first trimester of pregnancy since the rules for the newly pregnant mother apply because of the new tissue growth is also occurring after the stem cell transplantations treatment is performed.

### Factors that can Increase Stem Cell Growth

The following is recommended during the first three days following stem cell treatment of this invention.
1) Keep mind active and engaged, unless the patient is very tired (Pay heed to the signals that body sends patient. When tired, rest or sleep. When full of pep, do something challenging and hopefully uplifting).
2) Relax, sleep, pray, meditate, and be involved in creative, enjoyable activities. Such activities increase serotonin and melatonin that can help promote new stem cell growth.
3) Listen for a half hour to forms of classical music that patient enjoys. Music that provides a depth and complexity of rhythms, frequencies, timbre and internal integrity—such as is common to the “classics”—will bring about changes in nerves and brain regions connected to the inner ear. The result can be greater electrical activity and a synchronization of activity between various parts of the brain.
4) Drink 6-8 ounces of pure water. Water (not soft drinks or coffee) is important in cell-to-cell communication and stress reduction.
5) Eat selections from Dr. Steenblock's Regenerative Diet Program below. This diet is rich in fresh alkaline vegetables, moderate in poultry, fish and walnuts and low in saturated fat, total fat, and cholesterol. The diet does not include saturated fat-rich red meat, processed fruit, sweets, sugar-containing beverages, processed foods, or foods with additives, hormones, colors, preservatives, monosodium glutamate/vegetable hydrolyzed protein (MSG/VHP) or pesticides. (Pay attention to restrictions: Especially items #3 & #4 under the preceding section titled, “Factors that can inhibit stem cell growth and need to be avoided”)
6) Eat foods containing calcium, magnesium, potassium and B complex or that promote production of the mood-modulating neurotransmitter, serotonin. These nutrients help reduce stress and depression (Cognitive Therapy techniques work well to lessen or abolish stress) and may thus assist with stem cell proliferation. Serotonin-generating foods include squash, pumpkin, turnips, and celery (do not eat any brown spots on celery. They can promote free radical damage.) Calcium-rich foods include salmon, sardines, green leafy vegetables, collards, filberts, kale, kelp, mustard greens, prunes, sesame seeds, turnip greens, and watercress. Magnesium-rich foods include avocados, brewer's yeast, dulse, green leafy vegetables, salmon, sesame seeds, and watercress. Potassium foods include avocados, brewer's yeast, dulse, nuts, raisins, and winter squash. B complex foods include folic acid is in green leafy vegetables, asparagus, and spinach. Pyridoxine (Vitamin B6) is in Poultry, fish oil, vegetables, eggs, and sunflower seeds. Methylcobalamin (Vitamin B12) is in poultry, fish and fish oil. Refrain from using cyanocobalamin (A form of vitamin B12) contained in most B-complex vitamin pills.
7) Eats lots of DHA (docosahexaenoic acid) rich fish and seafood (See the chart below for seafood that is naturally low in mercury). This omega-3 fatty acid plays a role in nerve cell growth, cognition and modulating inflammatory responses.
8) Eat avocados, pumpkin seeds and sesame seeds. These foods contain tyrosine, a mood elevator. The processing of tyrosine in nervous tissue is associated with the growth and guidance of nerve pathways.
9) Eat foods containing zinc. Zinc-rich foods include eggs, turkey, sunflower seeds, and sesame seeds. Zinc is important in protein synthesis and nerve development and maintenance.
10) Eat kidney beans and hyacinth beans in moderate amounts. These beans contain a compound called “mannose-binding lectin” (FRIL) that helps sustain the viability of stem cells. Do not eat these if physician instructs patient not to.
11) Supplement with Ginseng. Ginseng has been reported to increase stem cell proliferation.
12) Eliminate all allergic foods. Determine your allergies through appropriate blood tests using IgE and IgG methodologies.

### Four Weeks After the Injection

After the stem cells have been given time to proliferate and migrate to where they are needed and proliferate they will then differentiate (“turn into”) into various cells such as new neurons, red blood cells, immune cells, etc. The following are prudent measures to implement about four weeks after the stem cell treatment of this invention.
1) Eat foods containing lots of vitamin A. Sources of Vitamin A include cod liver oil, fish oil, beet greens, watercress, kale, pumpkin, spinach, winter squash, and leafy lettuce.
2) Get 20 minutes a day of outdoors fresh air and moderate, indirect sunlight (refrain from the midday sun). Sunlight from the eyes may be directly converted to vitamin D in the brain. Vitamin D plays a role in the production of compounds such as brain derived neurotropic factor (BDNF) and nerve growth factor. These compounds can stimulate the growth of new brain cells. Brain-derived neurotrophic factor may also have a regenerative effect on the insulin producing cells in the pancreas (Brain-derived neurotropic factor has been shown to restore both pancreatic insulin and glucagon content in diabetic mice). It also plays a role in food intake and the regulation of glucose (blood sugar) metabolism by acting directly on the brain's appetite control center. If possible, patients should invest in full spectrum lighting
3) Get half an hour of moderate exercise each day. Start with stretching exercises for 10 minutes, then moderate aerobic exercise (move the limbs that can move) for 15 minutes, then relax for 5 minutes, gradually building greater endurance each week. Exercise increases brain-derived growth factor that plays a role in stem cell development. However, care must be taken not to cause stress by over-exertion. Physical therapy may begin or resume at four weeks post-transplantation.
4) Continue with Ginseng supplements. Ginseng also promotes stem cell differentiation
5) Avoid sports or other physical activities that may cause bodily injury since an injury might divert stem cells to newly damaged tissues instead of the target treatment site.
6) Avoid exposure to people who have colds, flu, etc. If the patient contracts such an infection, it may compromise stem cell activity or bring about bodily changes that divert stem cells from the intended treatment target.
7) Beginning six weeks post-transplantation, supplements with antioxidants may be taken. Antioxidants can help protect new stem cells and new neurons from the toxic effects of a compound called glutamate (Glutamate is produced in the body and can come from dietary sources like aspartame). This is especially important for those who are prone to worry and thinking the worst about situations, as this creates stress that weakens the body's ability to handle cell-damaging free radicals. Among the more potent antioxidants are Glutathione, Coenzyme Q10, N-acetyl cysteine (NAC), alpha lipoic acid, and vitamins A, C, and E.

### Dr. Steenblock's Regenerative Diet (Start Four Weeks After the Stem Cell Injection)

1) Resume eating antiproliferative flavonoids in moderation. These foods include onions, garlic, ginger, apples, berries, citrus, broccoli, cauliflower, Brussels sprouts, asparagus, almonds, and possibly some honey.
2) Continue to refrain from smoking, physical and emotional stress, alcoholic beverages, pollution, etc.
3) Follow Dr. Steenblock's Regenerative Diet Program, including exercise, stress-reduction, fresh air and moderate sunlight.
   a. Natural spring water or other contaminant-free water: 6-8 glasses a day will help promote intracellular communication. Avoid carbonated water, coffee and soft drinks.
   b. The Regenerative Diet is rich in fresh alkaline vegetables, moderate in poultry, fish and nuts and low in saturated fat, total fat, and cholesterol. The diet does not include saturated-rich red meat, fruit, sweets, sugar-containing beverages, processed foods, or foods with additives, hormones, colors, preservatives, monosodium glutamate/vegetable hydrolyzed protein (MSG/VHP) or pesticides. In addition, since milk products and grains can promote inflammation (and toxins to stem cells), these will need to be eliminated (Paleodiet orientation—For the rationale behind this go to http://14ushop.com/wizard/living-longer.html)
   c. Eat organic as much as possible. Since environmental toxins can be harmful to nerve cells and the activity of the cell's power-generating “factories” (mitochondria), a maintenance and regenerative diet needs to be as non-toxic as possible. While organic foods may still have some pesticide residues, certified organic foods are usually preferable to conventionally grown foods. Note that a recently published study (2004) found that fish farm sources of salmon, halibut, bass and trout appear to have more heavy metal contamination than fish from lakes and ocean sources. Fish is an important source of omega-3 fatty acids that protect brain cells from toxins and cell-damaging free radicals. However, if fish and poultry are eaten, they need to be as free of heavy metals such as methylmercury as possible.
   d. Eat fresh, whole foods as much as possible. If chewing is a problem, a blender can be used. Fresh foods provide the needed enzymes for more efficient digestion. Processed foods are made to last on the shelf for long periods of time and may therefore have preservatives, additives, colors, salts, and sugars.
   e. High Alkaline Diet: Improves immune function and protects against infection, inflammation and disease. The following are recommended: One serving of prunes every night before bedtime. (Is one of the highest sources of antioxidants). A 70% diet as raw vegetables and fruits: asparagus, beets, carob, carrots, cauliflower, celery, green beans, ripe olives, onions, parsnips, radishes, spinach, sprouts, string beans, watercress, chard, mustard greens, kale, carrots, leafy lettuce (no cabbage or iceberg lettuce) and fresh vegetables juices (no tomato juice). A Jerusalem artichoke twice a week assists with liver detoxification. Watercress, prunes and beet tops assist with elimination. A 30% the diet as: avocado, raw nuts, especially almonds and walnuts, fish, chicken or wild game.
   f. B vitamins and Homocysteine: Foods that contain natural folate, pyridoxine (B6) and methylcobalamin (B12) help reduce levels of homocysteine (A compound that can set the stage for damage to nerve cells and blood vessels). Homocysteine is a major cause of blood vessel wall injury and subsequent cardiovascular disease. Elevated levels of homocysteine have also been associated with cancer. Methylcobalamin is also important in the body's sleep/wake cycle (circadian rhythms) and to the production of the sleep hormone melatonin. Cyanocobalamin, the B12 form used in vitamins has a longer shelf life, but is not effective in improving brain function. Vitamin B12 can also directly block the nerve-damaging activity of glutamate and protect nerve cells in the retina against oxidative stress (free radical)-induced damage. Folic acid is in green leafy vegetables, asparagus, and spinach. Pyridoxine (Vitamin B6) is in Poultry, fish, fish oil, vegetables, eggs, and sunflower seeds. Methylcobalamin (Vitamin B12) is in poultry, fish, and fish oil.
   g. Fiber: According to the American Dietetic Association, the recommended daily intake of fiber for healthy adults is 20-35 g/day, with good sources being vegetables (Try to limit legume consumption such as potatoes and yams, as this causes blood sugar to rise precipitously and then drop sharply). Dietary fiber assists in lowering blood cholesterol levels and helps to normalize blood sugar and insulin levels, especially in patients with cardiovascular disease and Type 2 diabetes.
   h. Foods that contain antioxidants can assist the activity of the cell's power-production components (the mitochondria) and protect nerve cells from free radicals and such. Vegetables high in antioxidants include kale, spinach, Brussel Sprouts, alfalfa sprouts, broccoli, beets, and onions. Organic blueberries and red grapes are highly recommended, as they are rich in cell-protective compounds.
   i. Antioxidant Seasonings (Don't use for at least a month after the injection): Curcumin (curry), ginger, natural vanilla flavoring, Fenugreek, parsley, thyme, sage, rosemary, etc. can also be used as antioxidant flavorings to increase the healing benefits of the meal. However clove and cinnamon have been found interfere with energy production in the cell's mitochondria (energy producing factories of our cells) and are not recommended.
   j. Glutathione protects cells and neurons against free damage and is associated with improvement in diabetic retinopathy. Factors that increase and/or have a sparing effect on glutathione include moderate sunlight (vitamin D3), Fenugreek, riboflavin, aloe vera, ginger, vitamin E, Ginkgo biloba, pycnogenol, green tea, and vitamin C. The B vitamin riboflavin is also important [It plays an essential role in generating flavin adenine dinucleotide (FAD), a co-factor for an enzyme called glutathione reductase that helps in creating free-radical scavenging glutathione (26)] Note that various drugs, including Tylenol can deplete glutathione and therefore their use is discouraged.
   k. Foods that contain tryptophan should be included in planning one's diet. Typtophan is used to make the mood-modulating compound serotonin and the sleep hormone and antioxidant melatonin. One of the richest sources of tryptophan is turkey. It should be noted that reduced levels of tryptophan can impact niacin levels which is required by the mitochondria (Cellular powerhouses). Serotonin promoting foods include corn, squash, pumpkin, carrots, turnips, celery, and radishes.
   l. Eat smaller quantities of food on a more frequent basis (mini-meals) to help maintain stable blood sugar levels. Reduced caloric intake can increase the production of specific compounds (such as heat shock protein and brain-derived neurotrophic factor) that are important to nerve cell protection and blood sugar utilization and insulin activity.
   m. Glutamine intake increases the levels of glutathione in cells, which helps in mopping up cell-damaging free radicals. Glutamine also has anti-inflammatory effects and reduces cravings for sweets. Glutamine is in fish, parsley, and spinach.
   n. Acidophilus and bifidobacteria are helpful for promoting a healthy lining in the bowel and also prevents & treats leaky gut syndrome and constipation. These are available in supplement form at most health food stores.
   o. Fresh olives provide monosaturated fats that favorably influence various aspects of liver function, as well as those of skeletal muscles and the production of energy by cells in general. A healthy combination of fats would be 4 parts canola oil, 1 part fish oil, and 1 part olive oil.
   p. Prostacyclin enhancement: —Prostacyclin is made by cells that line blood vessels and helps open up these vessels and thus allow more oxygen-carrying red blood cells to flow through. Nutrients and herbs that help increase prostacyclin production include gamma linolenic acid (GLA), fish oil (EPA and DHA), and ginger.
   q. Growth Factors: Growth factor production is supported by copper SOD (superoxide dismutase) and copper-rich foods such as liver, Brazil nuts, raw oysters, and lobster. (Do not follow this recommendation if you are being treated for Parkinson's Disease or Cancer).

### Foods to Avoid:

1. Avoid red meats high in saturated fats. Diets high in animal fat or cholesterol can increase the risk of damage to cell membranes that can lead to cell die-off.
2. Avoid processed foods. They may contain additives, heavy metals, salt and sugar that increase free radical production (“oxidative stress”).
3. Avoid sweets and high glycemic index foods. Sugar-rich foods can increase bacteria and lead to increased infections. Sugar is a refined carbohydrate that quickly increases blood sugar levels followed by a surge in insulin (Blood sugar goes up, then can dip down dramatically These foods—called high glycemic index foods—can cause high blood sugar (hyperglycemia) and set the stage for producing cell-damaging free radicals.
4. Avoid food additives that contain or include the following: Artificial coloring (associated with cancer risk), aspartame (associated with nerve cell damaging glutamate), brominated vegetable oil (potential risk), BHA and BHT (potential risk), concentrated caffeine (associated with fibrocystic breast disease), carrageenan (associated with colon obstruction), corn syrup, dextrose, invert sugar and sucrose (sucrose is “table sugar” and has a high glycemic index which means it wrecks havoc with blood sugar levels), heptyl paraben (potential risk), hydrogenated vegetable oil (associated with immune impairment), hydrolyzed vegetable protein (HVP) (contains MSG—associated with promotion of excitatory neurotransmitters, nerve damage, burning sensations, headache), phosphoric acid and phosphates (associated with osteoporosis risk), propyl gallate (potential cancer risk), quinine (associated with birth defects), saccharin (associated with cancer risk), sodium chloride (associated with heart disease), sodium nitrite and sodium nitrate (associated with cancer risk), sulfur dioxide, sodium bisulfide (a bleach associated with allergies and B1 deficiency).
5. Avoid cigarettes and alcohol. —Cigarettes and alcohol increase hypoxia (lack of oxygen), cell-damaging free radical production and increased artery-wrecking homocysteine levels. Alcohol is toxic to new nerve cells. Alcohol is also a risk factor for high (hyperglycemia) and low (hypoglycemia) blood sugar levels.

### Treatment Results

Parents of eight cerebral palsy (CP) children consented to fill out questionnaires before the stem cell treatment, and one month, three months and six months following the transplant. All of these children had already been treated with various conventional therapies in previous years and their neurological status was considered stable by their parents and therapists.

The transplant procedure consisted of a simple subcutaneous intramuscular injection. The subjects were then observed for any adverse reactions for at least an hour and then released.

### The Questionnaire

The questionnaire included graft versus host symptoms as well as fine motor, gross motor, self-help, social and cognitive behaviors. There were 78 questions and space for comments. The first group of questions were about graft versus host symptoms. The second section was a dysfunction rating, i.e. unable to speak, poor attention span, etc. Pre-treatment ratings were compared with the final parent ratings. The rating scale for this section was:

| | |
|---|---|
| No Symptoms | 0 |
| Slight Symptoms | −1 |
| Moderate Symptoms | −2 |
| Severe Symptoms | −3 |

The third section was a rating for degree of improvement on behaviors such as speech, attention span, leg movement, etc. The rating scale for this section was

| | |
|---|---|
| No Improvement | 0 |
| Slight Improvement | 1 |
| Moderate Improvement | 2 |
| SignificantImprovement | 3 |

The improvement scores for the first month, third month and sixth month ratings were averaged for each of the eight children and a simple SPSS analysis was run, using mean comparisons in a paired-samples t-test.

### Results

### Graft Versus Host Reactions

Questions were taken from McKeena's report (McKeena D H, Wagner J E, McCullough J. Umbilical cord blood infusions are associated with mild reactions and are overall well tolerated. Poster Abstract, 9^{th} Annual Meeting of the International Society for Cellular Therapy, May 29-Jun. 1, 2003, Phoenix, Ariz.) on side effects from cord blood and included changes in heart rate and blood pressure, nausea, coughing, back pain, rashes, chills, excessive thirst, rapid breathing, headaches, etc.

In eight out of eight children, the parents reported no graft versus host symptoms from the stem cell transplants. One child experienced localized mild pain for three days where the injection was given. Three children needed more sleep in the weeks following the transplant and one child needed less sleep.

No other reactions were reported. This is highly significant, sincimmuno-suppressants, required for adult stem cell transplants, were not given. Purified umbilical cord derived stem cells were used, devoid of red and white blood cells and their antigens.

### Stem Cell Homing Challenges

Five of the eight children were rated by their parents as having moderate to significant improvement. The remaining three children had infections prior to or during the transplant. In the six months following the treatment, none of the children had infections again but showed only mild benefits in mobility and cognition.

### Internal Reliability

Several of the dysfunction symptoms in the second section of the questionnaire approached significance in a pre and post SPSS means comparison test for the parent ratings of the eight children. Symptoms that showed improvement, though not statistically significant, from the first pretreatment rating to the last post-treatment rating included:

Question 1: Sickness (0.19>p=0.05)
Question 18: Reduced muscle tone? (0.11>p=0.05)
Question 20: Eat unassisted? (0.17>p=0.05)
Question 25: Lethargy? (0.14>p=0.05)
Question 29. Poor memory? (0.19>p=0.05)
Question 30. Inability to speak? (0.17>p=0.05)
Question 31: Poor attention span? (0.11>p=0.05)
Question 39. Hearing problems? (0.18>p=0.05)
There were positive correlations between section 2 and section 3 in only 3 of the questions.

### Statistically Significant Improvements

The third section of the questionnaire was a parent rating for perceived improvement. Fifteen of the behaviors for all eight children, (who also received feedback from their pediatricians and physical therapists) were statistically significant (<p=0.05). These improvement ratings included:

1. Balance while standing (0.013<p=0.05)
2. Understanding (0.017<p=0.05)
3. Roll to Right/Left (0.018<p=0.05)
4. Leg Movement (0.019<p=0.05)
5. Hip Movement (0.019<p=0.05)
6. Muscle Tone (0.026<p=0.05)
7. Balance while sitting (0.026<p=0.05)
8. Stand Up (0.033<p=0.05)
9. Increased Sensation (0.033<p=0.05)
10. Hand Movement (0.035<p=0.05)
11. Vocabulary (0.043<p=0.05)
12. Thinking (0.044<p=0.05)
13. Transfer (0.045<p=0.05)
14. Balance in walking (0.046<p=0.05)
15. Arm Movement (0.050=p=0.05)
Not quite statistically significant was improvement in speech (0.074: >p 0.05).

Comments from the parents about their CP children were:
1) No perceivable improvement at all at three months. At four months, a slight improvement, perhaps due to physical therapy.
2) Her hands and feet are warmer, her eyes seem to try to focus. Hoping to see more improvement.
3) More energetic, looks better, interacts better, laughs at Barney on TV. Pediatrician commented that she is walking straighter, with greater balance.
4) Appears more aware, more attentive, better eye contract, less rigid movement, more balanced sitting, uses arms with more purpose, lower back arch less pronounced, back is less tense, movements more deliberate, responding faster to commands.
5) Using his sign language more consistently, putting together 2 signs to communicate of his own volition and being more creative with his limited language to tell us what he wants. His tantrums have decreased.
6) Slight improvement in putting arms straight up, more smooth movement, starting to talk more, understanding improving, less effort in helping him stand and walk, hearing improved, more sensitive to loud noises.
7) Increase in attention and awareness. Has shown improvements across the board including visual tracking and interactions with others. More alert, more strength in holding up head, smiles, listens to conversations, increased vocalizations such as laughing and crying and cooing, eye tracking, more interactions with people and a greater sense of happiness. Flexibility has improved, stronger with stepping, standing and crawling (with assistance).
8) Excellent mood, feels good. Stronger spine, muscular control, balance, increased range of motion in right arm, more able to use right hand. Able to isolate fingers on left hand and shoot a small ball through a hoop. More centered and stable.

### Conclusion

Multi-potent stem cells from umbilical cord blood have a number of distinct advantages.
1) No harm is done to the fetus or mother.
2) No loss of a potential life.
3) Infections are prevented by strict adherence to Quality control for cord blood communicable diseases, which has been established by the American Association of Blood Banks (AABB).
4) With embryonic stem cells, especially of unknown origin and viability, there is an increased risk of genetic mutations and tumors. However, umbilical cord derived stem cells as a part of umbilical cord blood, already has a track record of safety, having been used successfully in cancer patients for fifteen years.
5) With purified umbilical cord derived stem cells, there appears to be little or no risk of graft versus host disease. This may be because the umbilical cord derived stem cells are purified and separated from white and red cells and platelets (antigenic materials), and the stem cells themselves have a very low antigen expression on their surface membranes.
6) Chemotherapeutic immuno-suppressants are eliminated. Chemotherapy produces destructive neurological changes and cognitive decline for several years following a transplant.
7) Human umbilical cord derived stem cells are multi-potent cells that appear to be of clinical benefit for patients with cerebral palsy.

For neurological disorders (CP, MS, ALS, stroke, Alzheimer's Disease, Parkinson's Disease), stem cells appear to work best where there has been a sufficient “clean up” of infections and toxic conditions that appear to diminish the stem cell transplant clinical outcomes.

The results from this small sample and the limited time of follow-up (6 months) suggest that human umbilical cord derived stem cells are safe for children with cerebral palsy. The parent ratings also suggest that human umbilical cord derived stem cells may have a beneficial effect on ameliorating the signs and symptoms of cerebral palsy.

Children with cerebral palsy, autism, ADD/ADHD, and cognitive disabilities could potentially benefit from stem cells transplants using stem cells and neural progenitor cells derived from AABB certified human umbilical cord blood.

### SCOPE OF THE INVENTION

The above presents a description of the best mode contemplated of carrying out the present invention, and of the manner and process of making and using it, in such full, clear, concise, and exact terms as to enable any person skilled in the art to which it pertains to make and use this invention. This invention is, however, susceptible to modifications and alternate constructions from that discussed above which are fully equivalent. Consequently, it is not the intention to limit this invention to the particular embodiment disclosed. On the contrary, the intention is to cover all modifications and alternate constructions coming within the spirit and scope of the invention as generally expressed by the following claims, which particularly point out and distinctly claim the subject matter of the invention:

1. A method of treating a neurological disease comprising the step of administering to a patient a therapeutically effective amount of a composition including stem cells derived from an umbilical cord.

2. The method of claim 1 where the disease is cerebral palsy or traumatic brain injury.

3. The method of claim 1 where the composition includes growth factors mixed with the stem cells substantially immediately prior to administration of the composition to the patient.

4. The method of claim 3 where the growth factors include HGH (human growth hormone), G-CSF (granulocyte colony stimulating factor, GM-CSF (granulocyte macrophage colony stimulating factor, parathyroid (synthetic or natural) hormone, erythropoietin, stem cell factor, and LIF (leukemia inhibitory factor).

5. The method of claim 1 where growth factors are administered to the patient on, and at least substantially two days prior to, administering the composition.

6. The method of claim 1 where the composition includes blood plasma.

7. The method of claim 6 where the blood plasma is derived from the patient.

8. The method of claim 1 where the composition includes amniotic fluid or umbilical cord plasma or both.

9. The method of claim 1 where the composition is administered in the absence of any immuno-suppressant compound or chemotherapeutic agents.

10. The method of claim 1 where the composition is administered intravenously, intra-arterially, intramuscular, subcutaneously, intraperitoneally, intracutaneous, intralymphatically, or directly into the part of the patient's body being treated.

11. The method of claim 1 where at least substantially 85% of stem cells are selected from the group consisting of CD133+ umbilical cord stem cells, CD44− umbilical cord stem cells, CD45− umbilical cord stem cells, CD34−/45+ umbilical cord stem cells, CD34−/45− umbilical cord stem cells, and mesenchymal umbilical cord stem cells.

12. The method of claim 1 where the disease is cerebral palsy or traumatic brain injury in children under the age of substantially 13 and a single dose of the composition is administered, said dose including at least substantially 1 million stem cells that have been purified and expanded from AABB certified human umbilical cord blood, or Warton's Jelly, said stem cells being administered substantially in the absence of any immuno-suppressant compound or chemotherapeutic agents.

13. The method of claim 1 where the composition is purified to be substantially free of red and white blood cells and their antigens.

14. The method of claim 1 where the composition is administered intravenously by mixing with parenteral liquid flowing into a vein of the patient, said composition comprising umbilical cord stem cells where at least substantially 85% of cells are selected from the group consisting of CD133+ umbilical cord stem cells, CD44− umbilical cord stem cells, CD45− umbilical cord stem cells, CD34−/45+ umbilical cord stem cells, CD34−/45− umbilical cord stem cells, and mesenchymal umbilical cord stem cells, said umbilical cord stem cells being mixed with blood plasma derived from the patient substantially immediately prior to being administered intravenously and where the umbilical cord stem cells and the blood plasma are substantially free of red and white blood cells and their antigens.

15. The method of claim 1 where, prior to administering the composition, substantially eliminating in the patient extraneous infections and inflammations.

16. The method of claim 1 where, prior to administering the composition, substantially eliminating in the patient to the maximum extent possible heavy metals.

17. The method of claim 1 where, prior to administering the composition, substantially eliminating in the patient leaky gut syndrome and gut dysbiosis.

18. The method of claim 1 where, prior to administering the composition, administering to the patient growth factors.

19. The method of claim 1 where, at least five days prior to administering the composition and for at least substantially six months after administering the composition, substantially eliminating in the patient cortisone, steroids, glutamate (MSG), and alcohol and following an organic diet rich in anti-oxidants, and substantially free of pesticides, heavy metals, chemicals, food additives and food coloring agents.

20. The method of claim 1 where the patient is given physical therapy after administration of the composition.

21. The method of claim 1 where the patient is given chelation therapy prior to administration of the composition.

22. A method of treating cerebral palsy or traumatic brain injury comprising the step of administering to a patient a dose of at least 1 million umbilical cord stem cells of which at least substantially 85% of the cells are selected from the group consisting of CD133+ umbilical cord stem cells, CD44− umbilical cord stem cells, CD45− umbilical cord stem cells, CD34−/45+ umbilical cord stem cells, CD34−/45− umbilical cord stem cells, and mesenchymal umbilical cord stem cells.

23. The method of claim 22 where growth factors are mixed with the stem cells substantially immediately prior to administration of the dose.

24. The method of claim 22 where the composition comprises at least 1 substantially million umbilical cord stem cells, at least substantially 10 volume percent blood plasma, and a parenteral liquid including one or more amino acids.

25. A composition of matter comprising a mixture of human umbilical cord stem cells and growth factors mixed with the stem cells substantially immediately prior to administration of a single dose of the composition to a patient.

26. The composition of claim 25 where the growth factors include HGH (human growth hormone), G-CSF (granulocyte colony stimulating factor, GM-CSF (granulocyte macrophage colony stimulating factor, parathyroid (synthetic or natural) hormone, erythropoietin, stem cell factor, LIF (leukemia inhibitory factor).

27. The composition of claim 26 comprising at least substantially 1 million umbilical cord stem cells in said single dose and the growth factors are present in amounts and types that simulate growth factors present in umbilical cord plasma.

28. The composition of claim 25 comprising at least substantially 10 volume percent blood plasma and a parenteral liquid.

29. The composition of claim 25 where the parenteral liquid comprises physiological saline and water, Ringers Lactate, and substantially 5 weight percent dextrose in water.

30. The composition of claim 25 where the stem cells are derived from umbilical cord blood, Warton's Jelly, or mesenchymal (fibromuscular) stem cells derived from an umbilical cord wall, or a combination of any two or all three.

31. The composition of claim 25 where the stem cells are selected from the group consisting of CD133+ umbilical cord stem cells, CD44− umbilical cord stem cells, CD45− umbilical cord stem cells, CD34−/45+ umbilical cord stem cells, CD34−/45− umbilical cord stem cells, and mesenchymal umbilical cord stem cells.

32. The composition of claim 25 where the human growth factors are derived from blood plasma, said umbilical cord stem cells and blood plasma being substantially free of red and white blood cells and their antigens.

33. The composition of claim 32 where the blood plasma is derived from a patient undergoing medical treatment employing the composition

34. The composition of claim 25 where the human growth factors are growth factors contained in amniotic fluid or umbilical cord plasma or both, or which have been added.

35. A composition of matter comprising a mixture of human umbilical cord stem cells and blood plasma.

36. The composition of matter of claim 35 the blood plasma is derived from a patient undergoing medical treatment employing the composition and mixed with the stem cells substantially immediately prior to treatment.

37. The composition of claim 36 is a single dose including at least substantially 1 million umbilical cord stem cells of which at least substantially 85 percent of the cells are selected from the group consisting of CD133+ umbilical cord stem cells, CD44− umbilical cord stem cells, CD45− umbilical cord stem cells, CD34−/45+ umbilical cord stem cells, CD34−/45− umbilical cord stem cells, and mesenchymal umbilical cord stem cells.

38. The composition of claim 36 where the umbilical cord stem cells and the blood plasma are substantially free of red and white blood cells and their antigens.

39. The composition of matter of claim 36 including amniotic fluid or umbilical cord plasma or both.

40. The composition of claim 36 including a parental liquid and one or more amino acids.
